(19) United States
(12) Patent Application Publication
(10) Pub. No.: US 2007/0255231 A1
(43) Pub. Date: Nov. 1, 2007

(54) Vented directional delivery cannula with openings of different shape for use with flowable materials and method for use thereof

(75) Inventors: Jeffrey M. Gross, Memphis, TN (US); Jon Serbousek, Memphis, TN (US)

Correspondence Address: MARTIN & FERRARO, LLP, 1557 LAKE O'PINES STREET, NE, HARTVILLE, OH, 44632, US

(73) Assignee: SDGI Holdings, Inc.

(21) Appl. No.: 11/412,346

(22) Filed: Apr. 27, 2006

(51) Int. Cl. A61M 5/32

(52) U.S. Cl. 604/272

(57) ABSTRACT

In one preferred aspect, a cannula is provided that has openings of different shape to permit a surgeon to selectively control the distribution pattern of a flowable material through the cannula and into a patient. In another aspect, a method is provided for selectively controlling the distribution pattern of a flowable material through a cannula having openings of different shape into a patient.

### BACKGROUND OF THE INVENTION

1. Field of the Invention

The present invention relates generally to a vented cannula for delivery of flowable materials into a surgical site and a method for use thereof.

2. Description of the Related Art

A vertebral compression fracture in the human spine typically results in the partial collapse of the vertebral body. Instruments and methods exist to restabilize the vertebral body. One conventional vertebral compression fracture system is designed to deliver cement into the vertebral body with the cement material flowing out of the distal tip of a cannula. The flow of the cement will be in a pattern according to the path of least resistance, which is dependent on the native trabecula architecture and fracture plane in the immediate vicinity of the distal end of the cannula. Another cannula design has a uniform hole geometry along the length of the cannula and relies on a retrograde plunger to provide the force to extrude the cement radially outward along the length of the cannula. While such a cannula having a uniform hole geometry along the length may be adequate for filling the vertebral body with cement, there exists a need to have greater control of the flow and distribution of the cement into a vertebral body having a vertebral compression fracture.

The present disclosure seeks to use the inherent hydraulic force imparted to the cement via the delivery system proximal to the cannula to force the cement out along the cannula length in an optimized pattern to stabilize the vertebral body. The pattern of cement delivery is influenced by the number, size, shape, and radial and longitudinal distribution of openings along the length of the cannula.

The present invention utilizes principals inherent in the continuity equation, which in its basic form is: Q=VA, where Q is the flow rate, V is the velocity, and A is the area. By varying the dimension, number, and placement of openings along the length of the cannula, it is possible to selectively control the distribution of flowable materials through the cannula and into the patient.

### SUMMARY OF THE INVENTION

The present invention in one preferred embodiment includes a cannula for use in delivering a flowable material into the human tissue, the cannula including a proximal end, a distal end, a length therebetween, a mid-longitudinal axis, and an interior passage extending along the mid-longitudinal axis between the proximal and distal ends. The cannula includes a plurality of transverse openings between the proximal and distal ends. The transverse openings are in communication with the passage. At least two of the transverse openings are located proximate the distal end. The at least two transverse openings include at least one opening having a first shape and at least one opening having a second shape different than the first shape.

In another preferred embodiment, the present invention includes a method for delivering a flowable material into human tissue. The method includes providing a cannula having a proximal end, a distal end, a mid-longitudinal axis, and an interior passage extending along the mid-longitudinal axis between the proximal and distal ends, the cannula having a plurality of transverse openings between the proximal and distal ends, the transverse openings being in communication with the passage, the transverse openings including at least one opening having a first shape and at least one opening having a second shape different than the first shape. The method further includes inserting the cannula into the tissue with the transverse openings directed toward a location in the tissue where flowable material is desired to be delivered; delivering the flowable material through the passage of the cannula and out of the at least one transverse opening of the first shape into the tissue; and then delivering the flowable material through the passage of the cannula and out of the at least one transverse opening of the second shape into the tissue.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is a partial side elevation view of a cannula in accordance with one embodiment of the present invention inserted into the spine, the cannula having a longitudinal pattern of openings that increase in dimension toward the distal end of the cannula.

FIG. 2 is a partial cross-sectional side view of the cannula of FIG. 1.

FIG. 3 is a partial cross-sectional side view of a cannula having a longitudinal pattern of openings that decrease in dimension toward the distal end of the cannula in accordance with another preferred embodiment of the present invention.

FIG. 4 is a partial cross-sectional side view of a cannula having a longitudinal pattern of openings with different shapes in accordance with another preferred embodiment of the present invention.

FIG. 5 is a partial cross-sectional side view of a cannula having a longitudinal pattern of openings that increase and then decrease in dimension toward the distal end of the cannula in accordance with another preferred embodiment of the present invention.

FIG. 6 is a partial cross-sectional side view of a cannula in accordance with another preferred embodiment of the present invention, the cannula having a longitudinal pattern of openings that decrease in dimension toward the distal end of the cannula.

FIG. 7 is a partial cross-sectional side view of a cannula having a longitudinal pattern of openings that increase in dimension toward the distal end of the cannula in accordance with another preferred embodiment of the present invention.

FIG. 8 is a partial cross-sectional side view of a cannula in accordance with another preferred embodiment of the present invention, the cannula having a longitudinal pattern of openings that decrease in dimension toward the distal end of the cannula.

FIG. 9 is a partial cross-sectional side view of a cannula having a longitudinal pattern of openings that increase in dimension toward the distal end of the cannula in accordance with another preferred embodiment of the present invention.

FIG. 10 is a partial cross-sectional side view of a cannula having a longitudinal pattern of openings that increase in dimension toward the distal end of the cannula in accordance with another preferred embodiment of the present invention.

FIG. 11 is a side elevation view of a cannula having a longitudinal pattern of openings with various different shapes in accordance with another preferred embodiment of the present invention.

FIG. 12 is a side elevation view of a cannula having a longitudinal pattern of openings that increase in dimension toward the distal end of the cannula in accordance with another preferred embodiment of the present invention.

FIG. 13 is a transverse cross-sectional view of a cannula having a radial pattern of openings in accordance with another preferred embodiment of the present invention.

FIG. 14 is a transverse cross-sectional view of a cannula having a radial pattern of openings in accordance with another preferred embodiment of the present invention.

FIG. 15 is a transverse cross-sectional view of a cannula having a radial pattern of openings in accordance with another preferred embodiment of the present invention.

FIG. 16 is a transverse cross-sectional view of a cannula having a radial pattern of openings in accordance with another preferred embodiment of the present invention.

FIG. 17 is a partial side elevation view of a curved cannula in accordance with another preferred embodiment of the present invention.

### DETAILED DESCRIPTION OF THE PREFERRED EMBODIMENT

Reference will now be made in detail to the present preferred embodiments of the invention, examples of which are illustrated in the accompanying drawings.

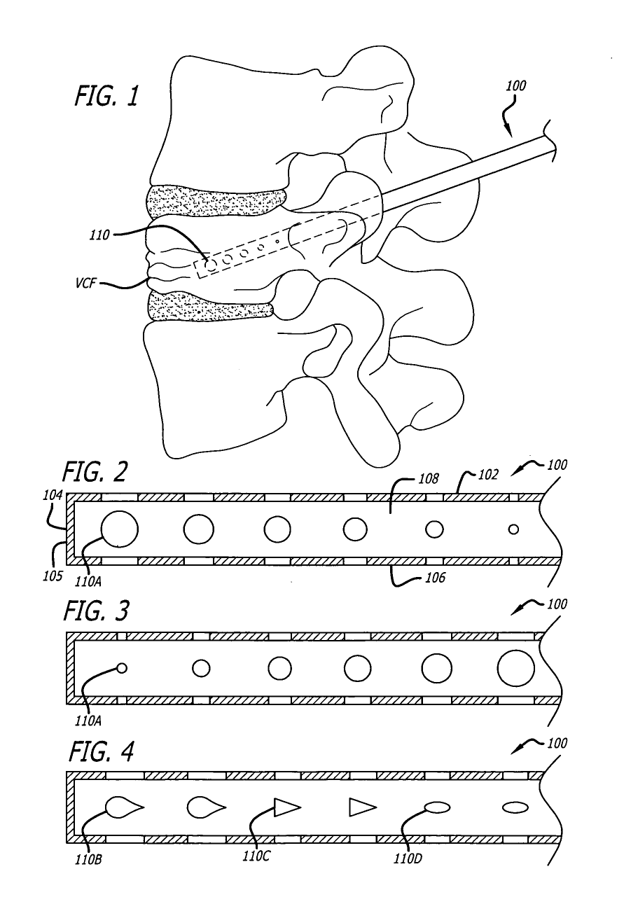

FIGS. 1-5 illustrate a cannula in accordance with one preferred embodiment of the present invention and is referred to by the reference number 100. Preferably, the cannula will include a plurality of openings of different quantity, size, and/or shape along the length of the cannula for allowing a user, such as for example a surgeon, clinician, or other qualified person, to selectively control the rate and distribution of a flowable material through the cannula and into a patient.

As shown in FIGS. 1 and 2, cannula 100 has a shaft 102, a proximal end (not shown), a distal end 104, a length from the proximal end to distal end 104, and a mid-longitudinal axis through the proximal and distal ends. Distal end 104 of cannula 100 includes a closed vertical front wall 105. It will be appreciated that the distal end of the cannula may be differently configured without departing from the scope of the present invention. For example, the distal end may be tapered or rounded to facilitate insertion of the cannula into the tissue of the patient. In such cases, the distal end may be frusto-conical, spherical, or elongated dome-shaped.

Referring to FIG. 2, shaft 102 includes a wall 106 defining a passage 108 along the mid-longitudinal axis. Wall 106 includes a plurality of transverse openings 110 therethrough each having a central longitudinal axis generally perpendicular to the mid-longitudinal axis of the cannula. It will be appreciated that one or more of the openings may have a central longitudinal axis that is at an angle other than perpendicular relative to the mid-longitudinal axis without departing from the scope of the present invention. Openings having an angled central longitudinal axis imparts directional control to the flow of material through the openings.

In each of the embodiments set forth below, openings 110 have a quantity, size, shape, and radial and longitudinal distribution configured to permit the selective control of the flow and distribution of a flowable material into a surgical site of a human body. Different patterns of openings and their interaction with a flowable material will be described below.

As shown in FIG. 2, cannula 100 has a plurality of openings 110A having a generally circular shape. Openings 110A have a size distribution that increases towards distal end 104 of cannula 100. A preferred rate of increase in the size distribution may include doubling the size of the opening immediately preceding the referenced opening. For example, openings 110A may each have a diameter that is twice the diameter of the opening immediately preceding to the referenced opening. It will be appreciated that other size distribution rates of increase or decrease may be included without departing from the scope of the present invention. For example, FIG. 2 shows the largest and most distal of openings 110A having a diameter that is approximately five times the diameter of the smallest and most proximal of openings 110A.

Openings 110A are circumferentially arranged about the mid-longitudinal axis at approximately 90-degree intervals. It will be appreciated by those of ordinary skill in the art that the openings may have a different radial distribution and number of radial openings without departing from the scope of the present invention. Further examples of radial distributions are described below with reference to FIGS. 13-16.

The distribution of increasing in size openings 110A in FIG. 2 permits a flowable material, such as but not limited to cements, bone void fillers, flowable carriers with growth factors, flowable carriers with analgesics, and combinations thereof, to be delivered in greater quantities to the surgical site closer to distal end 104. The circular shape of openings 110A facilitate a more rapid dispersal of flowable material because of a lower amount of resistance imparted on the material by the arcuate shape of the opening.

FIG. 3 shows openings 110A arranged such that the openings have a longitudinal size distribution that decreases in a direction towards distal end 104. Such a distribution is advantageous where the surgeon desires to deposit greater quantities of flowable material in a region more proximal relative to distal end 104.

FIG. 4 shows cannula 100 having a plurality of openings of different shapes. Openings 110B have a generally tear-dropped shape and are preferably positioned nearest to distal end 104 with the narrowest portion of the tear-drop oriented toward the proximal end of cannula 100. Openings 110C have a generally triangular shape and are preferably positioned proximal of openings 110B with the apex pointing toward the proximal end of cannula 100. Openings 110D have a generally oval shape and are preferably positioned proximal of openings 110C. Openings 110B, 110C, and 110D have a size distribution along the length of cannula 100 in a manner similar to that shown in FIG. 2 so that a larger quantity of flowable material will be distributed in a more distal region of the surgical site. Openings 110B, 110C and 110D have a shape distribution along the length of cannula 100 so that as the flowable material progresses towards distal end 104, the flow rate of the material exiting each shape is different along the length of cannula 100. This permits the surgeon to control the flow rate and distribution pattern of the material at a pre-selected location along the length of the cannula.

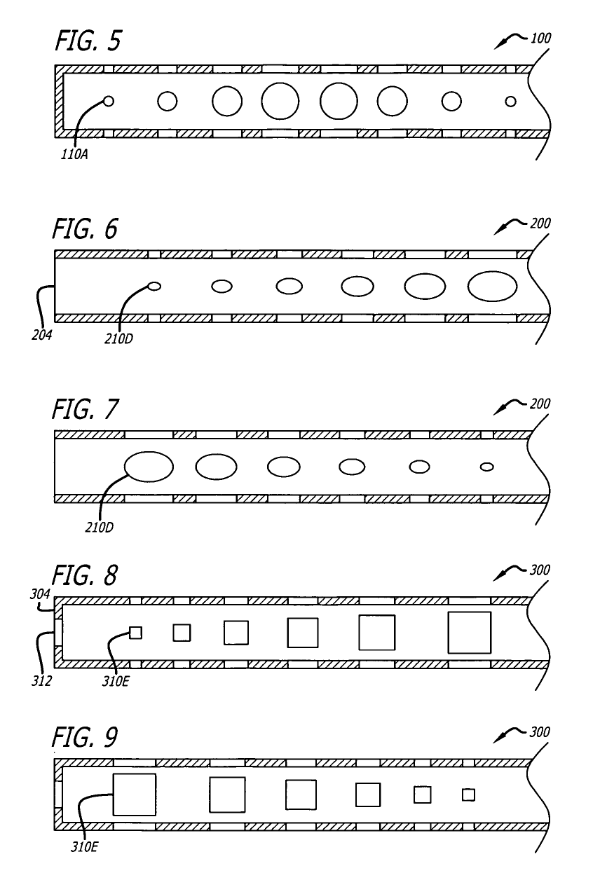

FIG. 5 shows cannula 100 having a variable size distribution of openings 110A. Openings 110A increase in size in a direction towards distal end 104 until they reach a maximum size, and then decrease to a lesser or minimal size proximate distal end 104. The size distribution shown in FIG. 5 is beneficial for selectively controlling the quantity of material at a location that is at a predetermined distance from the distal end of cannula 100.

A method utilizing the cannula shown in FIGS. 1 and 2 will now be described in relation to a surgical procedure in the spine. It will be appreciated that the cannula and method of the present invention may be used in other areas of the human body. For example, the cannula may be used for delivering a flowable material into tissue such as other bones of the human body, for example, a femur, or a region around the knee, or to deliver a therapeutic material to soft tissue.

The surgeon will preferably use a fluoroscope to obtain an image of the surgical site and map the region where the flowable material is desired to be deposited. Obtaining an image of the region where the flowable material needs to be deposited allows the surgeon to choose a pattern of openings sufficient to selectively deliver the flowable material more precisely into regions where the material is most needed. For example, should the surgeon desire to deposit a greater quantity of flowable material in a more proximal portion of the vertebral body, the surgeon could choose a pattern of openings with a decreasing size distribution such as illustrated in FIG. 3 so that the flowable material will first flow out of openings having greater size and with less material flowing out of the openings of lesser size towards the distal end of the cannula. Alternatively, if the surgeon desires to deposit more material in a distal region of the vertebral body, then the surgeon could choose a pattern of openings having an increasing size distribution such as shown in FIG. 2 so that the flowable material will be deposited more towards the distal region of the vertebral body. The surgeon may also choose a pattern of openings such that the shape of the openings differs along the length of the cannula so as to provide more selective control of the distribution of flowable material into particular regions of the vertebral body. In order to selectively control the quantity and rate of flowable material into particular regions, the surgeon could choose, for example, a pattern such as shown in FIG. 4. Once the surgeon chooses a pattern of openings that is suitable for the distribution pattern desired by the surgeon, the surgeon places a trocar into the lumen of the cannula. The trocar and cannula are connected at their proximal ends via a standard connection (not shown).

Next, the surgeon positions the trocar/cannula assembly adjacent to the surgical site and inserts the assembly into the tissue and through the vertebral body with the transverse openings directed toward a location in the surgical site where the flowable material is desired to be delivered. Once properly positioned, the surgeon removes the trocar leaving the cannula in position. A mechanical connection is made between the cannula and a material delivery system at the proximal end of the cannula. This system may use a direct coupling or a flexible tube may connect the cannula to the material delivery system. The material delivery system imparts a hydraulic force or mechanical force on the material to advance the flowable material through the cannula. The surgeon delivers the flowable material through the passage of the cannula so that the flowable material exits the openings into the areas desired by the surgeon. The flowable material is preferably delivered hydraulically through the cannula and into the patient. To hydraulically deliver the flowable material, the surgeon may, for example, use a plunger or other device that imparts a hydraulic force. Such a hydraulic device need not be directly connected to the cannula. For example, the hydraulic device may be connected to a flexible tube that is connected to the proximal end of the cannula.

Examples of flowable material useable with the cannula and method of the present invention include surgical bone cements, bone void filler material, or carriers that are synthetic, biologic, or a combination of synthetic and biologic, with adjunctive agents such as growth factors, analgesics, and anti-inflammatory agents. Examples of synthetic carriers include, but are not limited to, ceramics, bioresorbable materials, and polymers such as polyurethane and polylactic-co-glycolic acid (PLGA). Examples of biologic carriers include, but are not limited to, collagen or elastin. It will be appreciated that other therapeutic materials such as bone morphogenetic protein, hydroxyapatite, hydroxyapatite tricalcium phosphate, or an anti-microbial substance may be used with the cannula and method of the present invention without departing from the scope of the present invention.

Cannula 100 is made of a surgical grade material. Examples of suitable materials include, but are not limited to, metal such as stainless steel, titanium, and nitinol, carbon composites, and one or more plastic polymers. It will be appreciated that cannula 100 may be made of any combination of metal, plastic, carbon composite, or other materials suitable for the intended purpose. Preferably, cannula 100 is made as a rigid material. A rigid material facilitates insertion of the cannula into the patient. It will be appreciated by those of ordinary skill in the art that the cannula may be made of a flexible material, such as described below in relation to FIG. 17. A flexible material permits the surgeon to more selectively position the cannula once inserted into the surgical site.

In a preferred embodiment of the present invention, cannula 100 has a maximum length along the mid-longitudinal axis of the cannula that is approximately 15 cm between the distal end and the proximal end. The thickness of wall 106 of cannula 100 is preferably approximately in the range of 0.1 mm to 2 mm. The maximum transverse dimension of cannula 100 is preferably in the range of approximately 2 mm to 2 cm. The minimum transverse dimension of passage 108 is preferably larger than that of a standard needle, more preferably at least 2 mm. It will be appreciated that the dimensions set forth above may be varied without departing from the scope of the present invention. Unless otherwise noted, the above-described dimensions may be applicable to any one of the embodiments of the present invention.

Cannula 100 preferably has a generally circular cross-sectional shape transverse to the mid-longitudinal axis of the cannula. It will be appreciated by those of ordinary skill in the art that the cross-sectional shape of cannula 100 may be varied without departing from the scope of the present invention. For example, the cross section of the cannula may be square, rectangular, oval, or any other cross section suitable for the intended purpose. Additionally, cannula 100 may have an overall shape that is cylindrical, conical, or cylindrical with a conical distal end.

Referring now to FIGS. 6 and 7, a cannula in accordance with another preferred embodiment of the present invention is shown and referred to by the reference number 200. Cannula 200 is similar to cannula 100 except that distal end 204 is generally open. The open distal end of cannula 200 permits flowable material to be deposited into the surgical site distally along the mid-longitudinal axis of cannula 200. Cannula 200 includes a plurality of openings 210D that have a generally oval shape. The size distribution of openings 210D in FIG. 6 is similar to the size distribution of openings 110A in FIG. 3, decreasing in a direction towards distal end 204 along the length of cannula 200.

The size distribution of openings 210D in FIG. 7 is similar to the size distribution of openings 110A in FIG. 2, increasing in a direction towards distal end 204 of cannula 200. The general oval shape of openings 210D permit greater longitudinal control of flowable material therethrough as compared to the circular-shaped openings 110A shown in FIGS. 2 and 3 because of the greater resistance imparted upon the flowable material.

Referring now to FIGS. 8 and 9, a cannula in accordance with another preferred embodiment of the present invention is shown and referred to by the reference number 300. Cannula 300 is similar to cannula 100 except that cannula 300 includes a distal end opening 312 therethrough having a maximum dimension less than the maximum dimension of the passage immediately adjacent the distal end. Distal end opening 312 permits a quantity of flowable material to be deposited distally of cannula 300 along the mid-longitudinal axis of cannula 300. Distal end opening 312 has a dimension that is less than the open distal end of cannula 200 so that greater control of the distal deposit of material may be obtained.

As shown in FIG. 8, cannula 300 has a plurality of openings 310E that have a generally square shape. The size distribution of openings 310E is similar to that of FIG. 3 in that the size of openings 310E decrease in a direction towards distal end 304 along the length of cannula 300.

The size distribution of openings 310E in FIG. 9 is similar to the size distribution of openings 110A in FIG. 2, increasing in a direction towards distal end 304 of cannula 300. The general square shape of openings 310E permit greater longitudinal control of flowable material therethrough as compared to the circular-shaped openings 110A shown in FIGS. 2 and 3 because of the greater resistance imparted upon the flowable material.

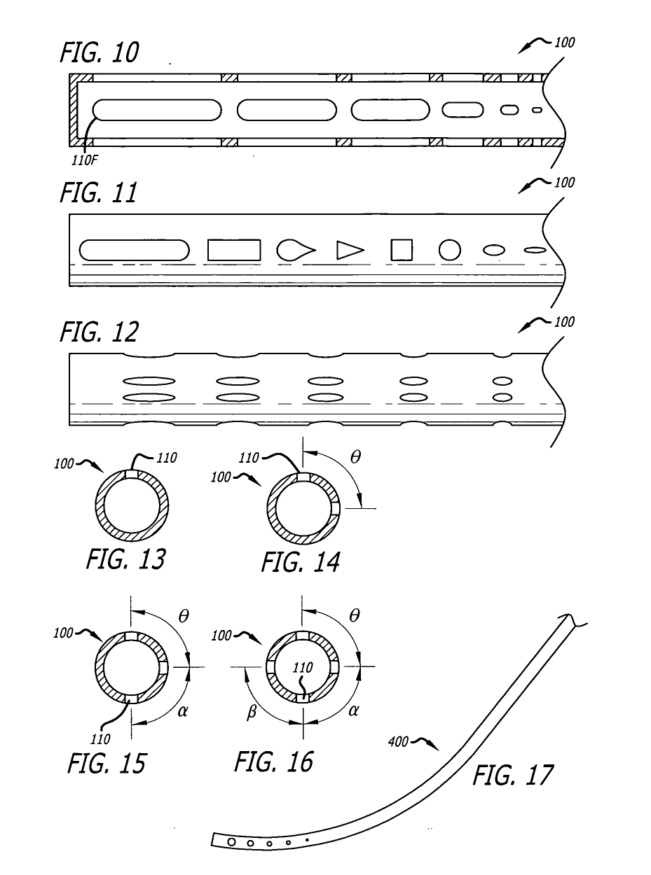

FIG. 10 shows cannula 100 having a plurality of openings 110F that each have a generally elongated oval shape. Openings 110F have a size distribution similar to that of FIG. 2, openings that increase in size in a direction towards distal end 104 along the length of cannula 100. The elongated oval shape of openings 110F provide better control for materials of higher viscosity as compared to the circular shape of openings 110A.

FIG. 11 shows cannula 100 having a plurality of openings that include a variety of shapes and sizes encompassing those described above in relation to FIGS. 2 to 10. The size distribution of openings 110 shown in FIG. 11 is similar to that of the size distribution shown in FIG. 2, increasing in a direction towards distal end 104 along the length of cannula 100. The shape pattern of the openings in FIG. 11 is different from that described in FIG. 2 so as to permit a surgeon to more selectively control the distribution of the flowable material through cannula 100 for a flowable material of a predetermined viscosity. For example, a surgeon may wish to select a cannula having a pattern of openings shown in FIG. 11 when the flowable material has a relatively high viscosity.

FIG. 12 shows cannula 100 having a plurality of openings 110D similar to that shown in FIG. 7. The size distribution shown in FIG. 12 is also similar to that of FIG. 7 in that the openings increase in size towards distal end 104 along the length of cannula 100. The pattern of openings shown in FIG. 12 is different from that of FIG. 7 in that a greater quantity of openings are radially positioned along any given portion of the length of the cannula. For example, as shown in FIG. 12, openings 110D may include a greater quantity of openings on the side portions of cannula 100 than on the upper and lower portions of cannula 100. This permits more flowable material to be deposited laterally of cannula 100 as opposed to above and below cannula 100.

FIGS. 13-16 illustrate radial placement of the transverse openings through the cannula. As shown in FIG. 13, openings 110 may extend through the wall of the cannula on only single side of the cannula in any particular transverse cross section along the length of the cannula.

As shown in FIG. 14, openings 110 may extend through the wall of a cannula in an upper portion and a side portion so that the central longitudinal axis of each opening intersects at a 90-degree angle θ. It will be appreciated that angle θ can be an angle other than 90 degrees without departing from the scope of the present invention. For example, angle θ may be in the range of 30 to 90 degrees.

As shown in FIG. 15, openings 110 may extend through the wall of a cannula in the upper, lower, and one side portion of the cannula so that the central longitudinal axis of each opening intersects at a 90-degree angle θ or α relative to the central longitudinal axis of another one of the openings. It will be appreciated that like angle θ, angle α can be an angle other than 90 degrees without departing from the scope of the present invention. For example, angle α may be in the range of 30 to 90 degrees.

As shown in FIG. 16, openings 110 may extend through the wall of the cannula in the upper, lower, and side portions so that the central longitudinal axis of each opening intersects at a 90-degree angle θ, α, or β relative to another one of the central longitudinal axis of the openings. It will be appreciated that like angles θ and α, angle β can be an angle other than 90 degrees without departing from the scope of the present invention. For example, angle β may be in the range of 30 to 90 degrees. The radial placement of openings shown in FIG. 16 is similar to that illustrated in FIGS. 2 to 10. It will be appreciated that the radial orientation of the openings may be at angles other than 90 degrees. For example, the central longitudinal axis of the openings may be positioned relative to one another in a transverse direction in the range of 30 to 90 degrees. It is also appreciated that more than four openings may exists along a radial orientation of the cannula at any given longitudinal length.

The radial placement of the openings may be such that they are coaxial relative to one another on opposite portions of the cannula, for example, such as shown in FIGS. 15 and 16, or may be axially offset along the length of the cannula. For example, one opening may have a central longitudinal axis that is vertical relative to the mid-longitudinal axis while another opening may have a central longitudinal axis that is at a 45 degree angle relative to the central longitudinal axis of the first opening and longitudinally displaced from the central longitudinal axis of the first opening. It will be appreciated that the central longitudinal axes of the openings may be at an angle to the mid-longitudinal axis of the cannula (i.e., intersecting the mid-longitudinal axis at an angle other than 90 degrees, or not intersecting the mid-longitudinal axis at all). Such a configuration imparts directional control to the flowable material. It will be appreciated that all of the openings may have the same axial angulation, selected openings may have different axial angulations, or all of the openings may have a different axial angulations depending upon the surgical environment intended for the use of the cannula.

If desired, the surgeon may use a cannula that is at least in part curved, flexible, or partially flexible so that the surgeon may move the distal end of the cannula into a region of the surgical site where the surgeon desires to deposit more material. An example of such a cannula is described below in relation to FIG. 17.

Referring now to FIG. 17, a cannula in accordance with another preferred embodiment of the present invention is shown and referred to by the reference number 400. Cannula 400 is similar to cannula 100 except that cannula 400 is made of a material that permits flexible placement of the distal end within a surgical site relative to the proximal end of the cannula. For example, cannula 400 can be made of a shape-memory material such as nitinol so that it is able to return to its original shape after being flexed. It will be appreciated that cannula 400 may include any one of the patterns of openings set forth above with respect to FIGS. 1-16.

There are numerous advantages of the present invention. For example, the surgeon may choose a pattern of openings that deposits the flowable material in a location where the material is most needed. This permits a more efficient use of the material and enhances the chances of a successful outcome of the surgical procedure. The surgeon may not only control the flow of the material, but also the distribution pattern of the material. The pattern of openings may be arranged so that the surgeon may deposit material in pre-selected locations in the surgical site based on an image of the surgical site. Such a distribution pattern is not achievable with conventional cannulas having uniform hole pattern or single hole at the distal tip of a cannula.

It will be appreciated by those of ordinary skill in the art that the present invention described above may take alternative forms without departing from the scope of the present invention. For example, for a cannula having a closed distal end such as that illustrated in FIGS. 2-5, the cannula may include one or more baffles in the passage at the distal end to channel the flowable material out of the openings for a more efficient distribution of flowable material. Additionally, one or more grooves may be included along the interior of the passage of the cannula to provide further guidance to the flowable material to one or more of the openings.

The cannula of the present invention preferably includes a single passage. It will be appreciated that that the cannula may have a plurality of passages without departing from the scope of the present invention. For example, different passages can lead to different sized or shaped openings along the length of the cannula if desired by the surgeon. Such a configuration would permit the surgeon to deliver different flowable materials to different areas of the surgical site.

Passage 108 shown in FIG. 2 has a generally uniform inner cross section along the length of the cannula. It will be appreciated that the dimension of the passage may be varied along the length of the cannula without departing from the scope of the present invention. For example, shaft 108 may have one or more tapered transitions along the length so that the cross sectional size of the passage transverse to the mid-longitudinal axis of the cannula may decrease in a direction towards distal end 104. A decrease in the size of the passage would decrease the flow rate of the material. It will be further appreciated that tapered transitions may be included so that the cross sectional size of the passage will increase in a direction towards distal end 104, thereby causing a increase in the flow rate towards the distal end. If desired, transitions may be included along the length of the passage so that the flow rate increases or decreases depending upon whether the passage is converging or diverging along the length of the cannula. Such a configuration will contribute to the flow control of the material through the cannula and out of pre-selected openings.

The cannula of the present invention may be disengageable from a handle so that the surgeon may choose amongst different patterns of openings suitable for the particular topography of the surgical site. Interchangeable distal cannula portions may be configured so that they may snap into or screw into a handle portion of the cannula.

The proximal end of the cannula may include an open end so that flowable material may enter passage 108 through the proximal end. Alternatively, it will be appreciated that the proximal end may be closed and that the flowable material may enter through one or more side ports of the cannula if so desired.

It will be appreciated by those of ordinary skill in the art that the pattern of openings may be varied depending upon the surgical environment that the cannula is intended to be used in. For example, the plurality of openings may be of a single shape such as illustrated in FIGS. 2, 6, 8, and 10. Where the surgical site has a more complex topography, different-shaped openings may be used in a single cannula to impart differing levels of resistance, for example, such as shown in FIGS. 4 and 11.

If the surgeon desires to deposit more flowable material towards the distal end of the cannula, then a size distribution may be used so that openings of larger size are positioned more distally while openings of lesser size are positioned more proximally, for example, such as shown in FIGS. 2, 7, 9, 10, and 11. The surgeon may desire to deposit more flowable material into the surgical site proximally than distally. Under such circumstances, the surgeon may then use a size distribution such that openings of larger dimension are located more proximally than distally. Such configurations are shown, for example, in FIGS. 3, 6, and 8.

The openings described above may have a configuration that is symmetrical, such as openings 110A, 110D, 110F, and 310E, or may be non-symmetrical such as openings 110B and 110C. The openings may form a non-arcuate polygonal shape such as openings 110C and 310E or may be completely arcuate such as openings 110A and 110D. Additionally the openings may form a single vertex or point such as openings 110B shown in FIG. 4, or may have several vertices such as openings 110C shown in FIG. 4. Preferably, openings with one or more vertices will be configured such that at least one of the vertices point in the proximal direction and the arcuate portion (if any) is oriented towards the distal direction such as shown in FIG. 4.

It will be appreciated that each embodiment of the cannula of the present invention set forth above is merely representational and that the features of each cannula may be interchanged or combined with the features of any of the other cannulas. For example only, instead of having a distal end opening 312, cannula 300 may have an open distal end such as shown in relation to cannula 200, or a closed distal end such as shown in relation to cannula 100, or may have a plurality of distal end openings without departing from the scope of the present invention. It will be further appreciated that where desired, the shape, size, and pattern of openings described in relation to FIGS. 2 to 16 may be useable with any of the cannulas described above without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A cannula for use in delivering a flowable material into human tissue, said cannula comprising:
a proximal end, a distal end, a length therebetween, a mid-longitudinal axis, and an interior passage extending along the mid-longitudinal axis between said proximal and distal ends, said cannula having a plurality of transverse openings between said proximal and distal ends, said transverse openings being in communication with said passage, at least two of said transverse openings being located proximate said distal end, said at least two transverse openings including at least one opening having a first shape and at least one opening having a second shape different than said first shape.

2. The cannula of claim 1, wherein said transverse openings vary in shape in a direction from said proximal end to said distal end.

3. The cannula of claim 1, wherein at lease one of said transverse openings is located proximal to said distal end of said cannula.

4. The cannula of claim 1, wherein at least two of said transverse openings are circumferentially offset from one another.

5. The cannula of claim 1, wherein at least two said transverse openings are arranged into axially spaced groups.

6. The cannula of claim 1, wherein said transverse openings include a plurality of axially aligned openings.

7. The cannula of claim 1, wherein said transverse openings are located only on one side of the mid-longitudinal axis along at least portion of the length of said cannula.

8. The cannula of claim 1, wherein said distal end is closed.

9. The cannula of claim 1, wherein said distal end is open.

10. The cannula of claim 1, wherein said distal end includes an opening having a maximum dimension transverse to the mid-longitudinal axis smaller than the maximum dimension of said passage transverse to the mid-longitudinal axis.

11. The cannula of claim 1, wherein said transverse openings have a shape including at least one of a circle, a ellipse, a square, a rectangle, a triangle, a slot, a slit, and a teardrop.

12. The cannula of claim 1, wherein at least a portion of said cannula is curved along the mid-longitudinal axis.

13. The cannula of claim 1, wherein said cannula is made of a rigid material suitable for surgical use.

14. The cannula of claim 1, wherein said cannula is made of a material suitable for surgical use and being at least in part flexible.

15. The cannula of claim 1, wherein said passage has a maximum cross sectional dimension transverse to the mid-longitudinal axis and at least one of said transverse openings having a maximum dimension less than the maximum cross sectional dimension of said passage.

16. The cannula of claim 1, wherein said tissue is bone.

17. The cannula of claim 1, wherein said tissue is a vertebra.

18. The cannula of claim 1, wherein said tissue is at least one of cartilage or other connective tissue.

19. The cannula of claim 1, wherein said cannula has an exterior surface that is generally smooth along a majority of the mid-longitudinal axis to facilitate linear insertion of said cannula into the tissue.

20. The cannula of claim 1, in combination with at least one of bone cement, bone void filler material, and a carrier with an adjunctive agent.

21. A method for delivering a flowable material into human tissue, said method comprising the steps of:
providing a cannula comprising a proximal end, a distal end, a mid-longitudinal axis, and an interior passage extending along the mid-longitudinal axis between the proximal and distal ends, the cannula having a plurality of transverse openings between the proximal and distal ends, the transverse openings being in communication with the passage, the transverse openings including at least one opening having a first shape and at least one opening having a second shape different than the first shape, inserting the cannula into the tissue with the transverse openings directed toward a location in the tissue where flowable material is desired to be delivered;
delivering the flowable material through the passage of the cannula and out of the at least one transverse openings of the first shape into the tissue; and
then delivering the flowable material through the passage of the cannula and out of the at least one transverse openings of the second shape into the tissue.

22. The method of claim 21, wherein a greater amount of the flowable material is delivered to the tissue through the transverse openings of the first shape than through the transverse openings of the second shape.

23. The method of claim 21, wherein the providing step includes providing the cannula with transverse openings varying in shape in a direction from the proximal end to the distal end.

24. The method of claim 21, wherein the providing step includes providing the cannula having an exterior surface being generally smooth along a majority of the mid-longitudinal axis of the cannula.

25. The method of claim 21, further comprising the step curving at least a portion of said cannula along the mid-longitudinal axis of the cannula.

26. The method of claim 21, wherein the flowable material is at least one of bone cement, bone void filler material, and a carrier with an adjunctive agent.

27. The method of claim 21, wherein the tissue is bone.

28. The method of claim 27, wherein the bone tissue is a vertebral body.

29. The cannula of claim 21, wherein the tissue is at least one of cartilage or other connective tissue.
